(19) United States
(12) Patent Application Publication
(10) Pub. No.: US 2010/0113577 A1
(43) Pub. Date: May 6, 2010

(54) ISOLATED NUCLEIC ACID MOLECULES CORRESPONDING TO MICRO RNA 145 (MIRNA-145) AND THEIR USE IN TREATING COLON CANCER

(76) Inventors: Bin Shi, Lansdale, PA (US); Laura Sepp-Lorenzino, Jenkintown, PA (US); Peter Linsley, Seattle, WA (US); Renato Baserga, Ardmore, PA (US)

Correspondence Address: MERCK, P O BOX 2000, RAHWAY, NJ, 07065-0907, US

(21) Appl. No.: 12/594,962

(22) PCT Filed: Apr. 7, 2008
(86) PCT No.: PCT/US08/04536

§ 371 Date: Oct. 7, 2009

Related U.S. Application Data
(60) Provisional application No. 60/922,850, filed on Apr. 11, 2007.

(51) Int. Cl. A61K 31/7088; C12Q 1/68

(52) U.S. Cl. 514/44 R; 435/6

(57) ABSTRACT

Provided herein are isolated nucleic acid molecule corresponding to miR145 that are useful in treating colon cancer. The disclosed miR145 nucleic acids specifically bind the 3′ UTR within endogenous IRS-I such as to suppress or inhibit colon cell proliferation.

### FIELD OF THE INVENTION

The present invention relates to novel small expressed (micro)RNA molecules associated with physiological regulatory mechanisms, and particularly useful in treating or down regulating cellular proliferative disorders such as colon cancer. More specifically, the invention relates to inhibiting growth of colon cancer cells by targeting IRS-1 via miRNA-145.

### BACKGROUND OF THE INVENTION

The insulin receptor substrate-1 (IRS-1) is one of the major substrates of both the type 1 insulin-like growth factor receptor (IGF-IR) and the insulin receptor (InR). IRS-1 plays an important role in cell growth and cell proliferation (1). IRS-1, especially when activated by the IGF-IR, sends an unambiguous mitogenic, anti-apoptotic and anti-differentiation signal (2, 3). IRS-1 levels are often increased in human cancer (4), and they are low or even absent in differentiating cells (1, 5, 6). Over-expression of IRS-1 causes cell transformation, including the ability to form colonies in soft agar and tumors in mice (7, 8). Transgenic expression of IRS-1 in the mammary gland of mice causes mammary hyperplasia, tumorigenicity and metastases (9). Conversely, down-regulation of IRS-1 (by antisense or siRNA procedures) reverses the transformed phenotype (10-12). The IRS proteins are conserved during evolution, and a gene described in Drosophila, called chico, is the equivalent of IRS-1 to 4 in mammalian cells. IRS proteins play an important role in cell size. Deletion of chico reduces fly weight by 65% in females and 55% in males (13). Mice with a targeted disruption of the IRS-1 genes are also smaller than their wild type littermates (14) and ectopic expression of IRS-1 increases rRNA synthesis and doubles cell size in cells in culture (7, 15). Thus, IRS-1 seems to play important roles in cell growth (cell size), cell proliferation and differentiation.

Most genes function by expressing a protein via an intermediate, termed messenger RNA (mRNA) or sense RNA. RNA interference (RNAi) describes a phenomenon whereby the presence of double-stranded RNA (dsRNA) of sequence that is identical or highly similar to sequence in a target gene mRNA results in inhibition of expression of the target gene. It has been found that RNAi in mammalian cells can be mediated by short interfering RNAs (siRNAs) of typically about 18-25 nucleotides (base pairs) in length. Functional siRNAs can be synthesized chemically or they can be formed endogenously through processing of long double strand RNA or transcription of siRNA encoding transgenes.

A type of genetic molecule barely on the radar screen of scientists a decade ago has emerged as a major player in cancer biology. Indeed, cancer initiation and progression can involve microRNAs (miRNA), which are small noncoding RNAs that can regulate gene expression. Their expression profiles can be used for the classification, diagnosis, and prognosis of human malignancies. Loss or amplification of miRNA genes has been reported in a variety of cancers, and altered patterns of miRNA expression may affect cell cycle and survival programs. Germ-line and somatic mutations in miRNAs or polymorphisms in the mRNAs targeted by miRNAs may also contribute to cancer predisposition and progression. First described in C. elegans more than a decade ago, >3,000 members of a new class of small noncoding RNAs, named microRNAs have been identified in the last 5 years in vertebrates, flies, worms, and plants, and even in viruses. See Calin G A, Dumitru C D, Shimizu M, et al. Frequent deletions and down-regulation of micro-RNA genes miR15 and miR16 at 13q14 in chronic lymphocytic leukemia. Proc Natl Acad Sci USA 99:15524-9 (2002). Functionally, it was shown that miRNAs reduce the levels of many of their target transcripts as well as the amount of protein encoded by these transcripts. See Lim L P, Lau N C, Garrett-Engele P, et al. Microarray analysis shows that some microRNAs down-regulate large numbers of target mRNAs. Nature 2005; 433:769-73 (3). Indeed some researchers have suggested that alterations in miRNA genes play a critical role in the pathophysiology of many, perhaps all, human cancers. Refer to Cancer Res.; 66(15): 7390-4 (2006).

For several miRNAs, the participation in essential biological processes has been proved, such as cell proliferation control (miR-125b and let-7), hematopoietic B-cell lineage fate (miR-181), B-cell survival (miR-15a and miR-16-1), brain patterning (miR-430), pancreatic cell insulin secretion (miR-375), and adipocyte development (miR-143). For reviews, see Harfe B D. MicroRNAs in vertebrate development. Curr Opin Genet Dev.; 15:410-5 (2005). These findings suggest that microRNA plays a major role in regulating gene activation.

MicroRNAs (miRNAs) are a family of short, non-coding RNAs that are thought to regulate post-transcriptional gene expression through sequence-specific base pairing with target mRNAs in a manner similar to RNAi. They are expressed in a wide variety of organisms ranging from plants to worms and humans.

Micro RNAs (miR5) are naturally-occurring 19 to 25 nucleotide transcripts found in over one hundred distinct organisms, including fruit flies, nematodes and humans. The characteristics of miRs have been summarized in several reviews (16-19). Briefly, miRs are cleaved from one arm of a longer endogenous double stranded precursor (70-100 nt in length) by Dosher and Dicer enzymes (RNase DI family). They are transcribed by RNA polymerase II (20) as long primary transcripts (pri-miRNAs), which are cropped and cleaved to produce the pre-miR and the mature miR (21). They are complementary to genomic regions and one of their modes of action is to bind to the 3′ untranslated regions of mRNA (3′UTR), inhibiting translation (the target mRNA levels remain unchanged). They can function also by cleaving a target mRNA, in which case the miR may target sequences outside the 3′UTR (18). miRs play crucial roles in eukaryotic gene regulation, especially in development and differentiation (22-25). A few reports have tied miRs to cancer (26-30). Targets of miRs can be obtained from the database (see below), although it is understood that the presumed targets have to be validated experimentally. None of the published report however have demonstrated a link between miR145 and IRS-1 as a means of treating colon cancer, wherein the miR145 specifically targets a region within the 3′ untranslated region (UTR) of IRS-1.

The inventors herein for the first time demonstrate a direct link between miR145 and IRS-1 as a means of treating colon cancer by specifically targeting endogenous IRS-1 via miR145. Specifically, the inventors demonstrate the use of synthetic oligonucleotides (oligos) corresponding to or substantially identical to wild type miR145 to specifically down-regulate IRS-1 in human colon cancer cells and that its effect is slightly less pronounced than the effect of an siRNA against IRS-1. While the siRNA causes a down-regulation of IRS-1 mRNA, miR145 does not, indicating that the effect is probably on translation. A reporter gene carrying the 3′UTR or the miR145 binding sites of IRS-1 is also down-regulated by miR145, while an IRS-1 cDNA without its 3′UTR is not affected. Finally, an expression plasmid expressing a hairpin precursor miR145 also down-regulated IRS-1 when transfected into colon cancer cells. Although siRNA is more effective than miR145 in down-regulating IRS-1 levels, miR145 and siRNA have similar inhibitory effects on the growth of colon cancer cells in culture; in fact, in some experiments miR145 was more potent than siRNA in inhibiting cell proliferation. This is probably because miRs target multiple proteins along the same pathway (31, 32). Indeed, miR145 targets also the IGF-IR (see below). Taken together, the results detailed herein demonstrate that miR145 targets the 3′UTR of IRS-1, and that the targeting has a profound effect on the growth of human colon cancer cells. This is the first demonstration of a specific miR targeting a transduction molecule of the IGF-IR/insulin receptor signaling pathway (IRS-1). Its inhibition of growth in human cancer cells in culture is compatible with the well known ability of IRS-1 to stimulate cell proliferation and transformation.

### SUMMARY

The insulin receptor substrate-1 (IRS-1), a docking protein for both the type 1 insulin-like growth factor receptor (IGF-IR) and the insulin receptor, are each known to transmit a proliferative, anti-apoptotic and anti-differentiation signal. We show here that one of the miR5, miR145, whether transfected as a synthetic oligonucleotide or expressed from a plasmid, causes down-regulation of IRS-1 in human colon cancer cells. IRS-1 mRNA is unaffected by miR145, while it is down-regulated by an siRNA targeting IRS-1. Targeting of the IRS-13′UTR by miR145 was confirmed using a reporter gene (luciferase) expressing the miR145 binding sites of the IRS-1 3′ UTR. In agreement with the role of IRS-1 in cell proliferation, the invention demonstrates that treatment of human colon cancer cells with miR145 causes growth arrest comparable to the use of an siRNA against IRS-1. Taken together, these results identify miR145 as a micro RNA that down-regulates IRS-1, and inhibits the growth of human cancer cells.

In one aspect, the present invention relates to an isolated nucleic acid molecule comprising:

(a) a nucleotide sequence as shown in one of SEQ ID NO:1 or 2;

(b) a nucleotide sequence which is the complement of (a),

(c) a nucleotide sequence which has an identity of at least 80%, preferably of at least 90% and more preferably of at least 99%, to a sequence of (a) or (b) and/or

(d) a nucleotide sequence which hybridizes under stringent conditions to a sequence of (a), (b) and/or (c).

Preferably the identity of sequence (c) to a sequence of (a) or (b) is at least 90%, more preferably at least 95%.

In a preferred embodiment the invention relates to miRNA molecules and analogs thereof, to miRNA precursor molecules and to DNA molecules encoding miRNA or miRNA precursor molecules.

The isolated nucleic acid molecules of the invention preferably have a length of from 18 to 100 nucleotides, and more preferably from 18 to 80 nucleotides. It should be noted that mature miRNAs usually have a length of 19-24 nucleotides, particularly 21, 22 or 23 nucleotides. The miRNAs, however, may be also provided as a precursor which usually has a length of 50-90 nucleotides, particularly 60-80 nucleotides. It should be noted that the precursor may be produced by processing of a primary transcript which may have a length of >100 nucleotides.

The nucleic acid molecules may be present in single-stranded or double-stranded form. The miRNA as such is usually a single-stranded molecule, while the mi-precursor is usually an at least partially self-complementary molecule capable of forming double-stranded portions, e.g. stem- and loop-structures. DNA molecules encoding the miRNA and miRNA precursor molecules are also within the scope of the invention. The nucleic acids may be selected from RNA, DNA or nucleic acid analog molecules, such as sugar- or backbone-modified ribonucleotides or deoxyribonucleotides. It should be noted, however, that other nucleic analogs, such as peptide nucleic acids (PNA) or locked nucleic acids (LNA), are also suitable.

In another aspect of the invention, the isolated nucleic acid molecule is an RNA- or DNA molecule, which contains at least one modified nucleotide analog, i.e. a naturally occurring ribonucleotide or deoxyribonucleotide is substituted by a non-naturally occurring nucleotide. The modified nucleotide analog may be located for example at the 5′-end and/or the 3′-end of the nucleic acid molecule. See pending application Ser. No. 60/861,369 ('369), filed Nov. 28, 2006, which provides additional information as to various other chemical modifications that may be made to the nucleic and molecules described herein. For purposes of the present invention, the contents of Ser. No. 60/861,369, is incorporated by reference herein in its entirety.

In certain embodiments, nucleotide analogs are selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. In preferred sugar-modified ribonucleotides the 2′-OH-group is replaced by a group selected from H, OR, R, halo, SH, SR, NH₂, MIR, NR₂ or CN, wherein R is C₁-C₆ alkyl, alkenyl or alkynyl and halo is F, Cl, Br or I. In preferred backbone-modified ribonucleotides the phosphoester group connecting to adjacent ribonucleotides is replaced by a modified group, e.g. of phosphothioate group. It should be noted that the above modifications may be combined. As used herein, the siRNA molecules need not be limited to those molecules containing only RNA, but may further encompasses chemically-modified nucleotides and non-nucleotides. WO2005/078097; WO2005/0020521 and WO2003/070918 detailing various chemical modifications to RNAi molecules, wherein the contents of each reference are incorporated by reference in its entirety.

In certain embodiments for example, the short interfering nucleic acid molecules may lack 2′-hydroxy (2′-OH) containing nucleotides. The RNA molecules of the invention can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase DI or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). The long dsRNA can encode for an entire gene transcript or a partial gene transcript.

The nucleic acid molecules of the invention may be obtained by chemical synthesis methods or by recombinant methods, e.g. by enzymatic transcription from synthetic DNA-templates or from DNA-plasmids isolated from recombinant organisms. Typically phage RNA-polymerases are used for transcription, such as T7, T3 or SP6 RNA-polymerases.

The invention also relates to a recombinant expression vector comprising a recombinant nucleic acid operatively linked to an expression control sequence, wherein expression, i.e. transcription and optionally further processing results in a miRNA-molecule or miRNA precursor molecule as described above. The vector is preferably a DNA-vector, e.g. a viral vector or a plasmid, particularly an expression vector suitable for nucleic acid expression in eukaryotic, more particularly mammalian cells. The recombinant nucleic acid contained in said vector may be a sequence which results in the transcription of the miRNA-molecule as such, a precursor or a primary transcript thereof, which may be further processed to give the miRNA-molecule. Various methods of delivering the mature miR145 or its pre-cursor are available to one skilled in the art. Exemplary references that detail methods of synthesis and delivery of DNA or RNA based vectors for transcribing long mRNA include U.S. Pat. No. 6,573,099, WO 00/44914, WO 01/36646, WO 01/75164 & WO 00/44895, the contents of which are incorporated herein by reference in its entirety.

Use of recombinant minicells for in vitro and in vivo targeting of the RNAi constructs are also included. See, for example, WO2005079854, WO2006021894, US2005222057 including references cited therein, the contents of each of which is incorporated by reference herein in its entirety.

In another embodiment, the invention provides a method of reducing expression of a target gene in a cell comprising obtaining at least one siRNA of the invention, and delivering the siRNA into the cell.

In general, the claimed nucleic acid molecules may be used as a modulator of the expression of genes which are at least partially complementary to said nucleic acid. Further, miRNA molecules may act as target for therapeutic screening procedures, e.g. inhibition or activation of miRNA molecules might modulate a cellular differentiation process, e.g. apoptosis.

Further, the invention relates to diagnostic or therapeutic applications of the claimed nucleic acid molecules. From a therapeutic standpoint, the claimed nucleic acid molecules may be used as modulators or targets of developmental processes or disorders associated with developmental dysfunctions, such as cancer. For example, miR145 functions as a tumor repressor but in certain pathologies its expression is down regulated. Thus, in those circumstances, expression or delivery of the RNAs or analogs or precursors thereof to tumor cells may provide therapeutic efficacy, particularly against colon cancer.

Furthermore, existing miRNA molecules may be used as starting materials for the manufacture of sequence-modified miRNA molecules, in order to modify the target-specificity thereof, e.g. an oncogene, a multidrug-resistance gene or another therapeutic target gene. The novel engineered miRNA molecules preferably have an identity of at least 80% to the starting miRNA, e.g. as depicted in SEQ ID Nos. 1 & 2. Further, miRNA molecules can be modified, in order that they are symetrically processed and then generated as double-stranded siRNAs which are again directed against therapeutically relevant targets, e.g., IRS-1.

In another aspect, the miRNA molecule disclosed herein or derived from those disclosed herein may be used for tissue reprogramming procedures, e.g. a differentiated cell line might be transformed by expression of miRNA molecules into a different cell type or a stem cell.

For diagnostic or therapeutic applications, the claimed RNA molecules are preferably provided as a pharmaceutical composition. This pharmaceutical composition comprises as an active agent at least one nucleic acid molecule as described above and optionally a pharmaceutically acceptable carrier.

Methods to limit or eliminate off-target silencing are also within the scope of the invention. Methods of modifying a polynucleotide for reducing off target silencing in RNA interference are known. See, for example, US 2005/0223427, the contents of which are incorporated by reference herein in its entirety.

The administration of the pharmaceutical composition may be carried out by known methods, wherein a nucleic acid is introduced into a desired target cell in vitro or in vivo.

### BRIEF DESCRIPTION OF THE DRAWINGS

FIG. 1. Micro RNAs and the structure of the IRS-1 gene. Panel A: list of the 8 miRs more likely to target IRS-1 according to the database. Panel B: Schematic structure of the pre-mRNA of IRS-1. Panel C: Schematic structure of mature IRS-1 mRNA and location of the two probable binding sites on the 3′UTR of IRS-1 cDNA for miR 145, one is starting from nucleotide (nt) 1 of the 3′UTR and the other is from nt 173 of the 3′UTR. The nucleotide sequences at the bottom of Panel C illustrate the predicted base-pairing between miR145 oligo (top strand) and the 3′UTR of IRS-1 (bottom strand). The sources from the database are listed in Methods and Materials.

FIG. 2. miR145 and IRS-1 levels in selected cell lines. Panel A: Absolute levels of miR145 in parental HCT116 cells and HCT116-Dicer-KO cells (50 ng total RNA samples each) detected by TaqMan. The miR145 levels were too low in both cells to be detected by Northern blots. Panel B: levels of IRS-1 in selected cell lines. Western blots with antibodies to IRS-1 and GAPDH (to monitor protein loading) from lysates of the cell lines indicated above the lanes. R+ and R12 cells are mouse embryo fibroblasts known to express normal amounts of IRS-1. BT-20 cells are human mammary cancer cells that do not express IRS-1 (8, negative control). Panel C. miR145 oligos transfected and detected in KO cells. Synthetic oligos for miR145 and miR148a were transfected into both HCT and DLD1 KO cells, and after 24 hrs, a Northern blot was done to detect the presence of miR145. The blot was exposed for 6 min (lower bands), or for 3 hrs (upper bands). miR145 cannot be detected in cells transfected with ds-miR148a oligos, which served as negative control.

FIG. 3. IRS-1 levels were down-regulated by the transfection of synthetic miRs' oligonucleotides. Transient transfection of the indicated miRs' synthetic oligonucleotides into parental HCT116 cells, HCT116-Dicer-KO and DLD1-Dicer-KO cells. The levels of IRS-1 were determined by western blot 96 hrs after transfection. miR148a oligos do not down-regulate IRS-1 in these experiments and could be used as negative control. GAPDH was used again to monitor loading.

FIG. 4. Effect of miR145 and siRNA on IRS-1 levels at various times after transfection. Panel A: repeated experiments in which synthetic miR145 oligos were transfected into HCT116-Dicer-KO cells. Western blot for IRS-1 after 96 hrs. The middle band shows the unchanged levels of the control GAPDH. The lower band shows that the α+β subunits of the IGF-IR is also affected. Panel B: effect of miR145 oligos and siRNA against IRS-1 on IRS-1 levels. The left panel shows that even with siRNA, IRS-1 levels were still high 24 hrs after transfection. 5 days after transfection, siRNA was shown to be more effective than miR145 in down-regulating IRS-1 levels.

FIG. 5. Levels of IRS-1 mRNA in KO cells transfected with miR145 oligos or siRNA against IRS1. The levels of IRS-1 mRNA in KO cells were determined by TaqMan real-time PCR at 24 and 96 hrs after transfection of miR145 oligos or siRNA/IRS-1.

FIG. 6. The 3′UTR of IRS-1 causes the down-regulation of a reporter gene. psiCHECK2 contains the luciferase reporter gene. Four constructs were made in which the 3′UTR of the reporter was replaced by: binding site no. 1 (Site #1), binding site no. 2 (Site # 2), both binding sites (Site 1+2) or the entire 3′UTR of IRS1 (see FIG. 1). The 5 plasmids were transfected with or without the miR145 oligos, and luciferase levels were determined after 48 hrs (for corrections due to transfection efficiency, see Methods and Materials). Data was generated from three repeated experiments.

FIG. 7. miR145 did not down-regulate an IRS-1 without its 3′UTR. A truncated IRS-1 cDNA (see text) lacking its 3′UTR was transfected with miR145 oligos or an siRNA against IRS-1 as indicated above the lanes. After 96 hrs, the IRS-1 levels were determined by western blots (the truncated IRS-1 is identified by its shorter length). A densitometric quantitation is shown below.

FIG. 8. Expression of miR145 in the pSuper plasmid. Panel A shows a quantitation of mature miR145 expression by Taqman after transfection of the indicated cell lines (parental HC116, HCT116-KO and 293 FT cells) with different constructs (miR145 hairpin with 20, 40, 80 or 160 nucleotides of flanking genomic sequences (see text). Panel B: Northern blot of miR145 in KO cells transfected with the different constructs of pSuper described in panel A. There is a good correlation between Taqman and Northern blots. Panel C: The pSuper plasmid expressing the miR145 hairpin with 20 nucleotides flanking sequences down-regulated IRS-1 protein levels 96 hrs after transfection.

FIG. 9. Effect of miR145 on the growth and morphology of KO cells. HCT116 KO cells were transfected with synthetic oligos (as indicated in the figure) or with siRNA/IRS1 (mock transfected cells served as the control). The plates were examined 4 days after transfection. Upper row: picture of the stained plates. 2^{nd} row: picture of the plates at 20× magnification. 3^{rd} row: levels of IRS-1 as determined by western blot. Last row: levels of GAPDH.

FIGS. 10 & 10A: Schematic structure of messenger RNA of IRS-1 and potential binding sites e.g., #1 and #2 of miR145 within 3′ UTR.

### DETAILED DISCUSSION

The contents of each reference cited herein is incorporated by reference herein in its entirety.

### Experimental Procedures

Cells—Colorectal cancer cell lines HCT116-Dicer-KO#2 and DLD1-Dicer-KO#4 were ki provided by Dr. Bert Vogelstein (33), and the parental cells HCT116 and DLD1 were from ATCC (Manassas, Va.). Both lines are derived from human colorectal adenocarcinomas cell lines. All the cells were cultured in McCoy's 5A medium supplemented with 10% fetal bovine serum and penicillin/streptomycin. In the Dicer-KO cells, the exon 5 of the Dicer gene encoding helicase is replaced by a neoR gene. BT-20, a human breast cancer cell line, was from ATCC and grew in DMEM/F12 medium supplemented with 10% calf serum, L-glutamine, and penicillin/streptomycin. R+ and R12 cells (34) were generated from R− cells, which are 3T3-like mouse embryonic fibroblasts (MEFs) with a targeted disruption of endogenous IGF-IR genes. R+ and R12 cells are R− cells stably transfected with a plasmid expressing human IGF1R. R+ had 300-fold more IGF1R (9×10⁵ receptors) than R12 (3×10³ receptors). R+ cells grow in serum-free medium supplemented solely with IGF-I, whereas R12 do not. Both cell lines were cultured in DMEM+10% FBS+ penicillin/streptomycin medium.

Double-strand oligos and Transfection—The ds-oligos miR145, miR148a, miR207, and miR154 as well as miR negative control were purchased from Dharmacon (Chicago, Ill.). SmartPool siRNA against human IRS1 was purchased from Upstate (Millipore, Charlottesville, Va.). The ds-oligos (50 nM) and plasmid DNAs (800 ng/ml) were transfected into parental and Dicer-KO cells by Lipofectamine-2000 (Invitrogen, Carlsbad, Calif.) in 6-well plates according to manufacturer's instruction.

TagMan Real-Time RT-PCR: Messenger RNAs of IRS1 were extracted using RNeasy Mini kit (Qiagen, Valencia, Calif.). miRNAs were extracted using Micro RNA Isolation Kit (Stratagene, LaJolla, Calif.) or mirVana miRNA Isolation kit (Ambion, Austin, Tex.). Primers and probes specific for human IRS1 and internal control 18S rRNA were purchased from Applied Biosystems (ABI, Framingham, Mass.). TaqMan One-step RT-PCR Master Mix Reagents Kit (ABI, Roche, Branchburg, N.J.) was used to detect IRS1 mRNA. Amplification and detection was performed using 7900HT Sequence Detection System (ABI), using 40 cycles of denaturation at 95° C. (15 s) and annealing/extension at 60° C. (60 s). This was preceded by reverse transcription at 50° C. for 30 mM and denaturation at 95° C. for 10 min. To quantitate mature miRNA, TaqMan® MicroRNA Assays kits were purchased from ABI to detect miR145 (Cat#4373133) and a control miR(RNU6B, Cat# 4373381). It is a two-step protocol requiring reverse transcription (Cat#4366596) with a miRNA-specific primer, followed by real-time PCR with TaqMan® probes (Cat#432-4018). The assays targets only mature microRNAs, not their precursors, ensuring biologically relevant results. The fold change of target gene in treatment groups relative to mock treated samples were calculated according to ABI's Relative

Quantification Methodology: The absolute miR145 levels in parental HCT116 and HCT116-Dicer-KO cells were also calculated according to a standard curve of miR145 (Dharmacon's hsa-miR-145 ds-oligo served as the standard). For details, refer to ABI's user's bulletin “Relative Quantitation of Gene Expression: ABI PRISM 7700 Sequence Detection System: User Bulletin #2: Rev B”.

Northern blot analysis: Northern blots were performed to confirm the expression levels of miR145. Ten to 20 μg of total RNA were separated on a 15% denaturing TBE-urea mini-gel (Invitrogen, Carlsbad, Calif.) and then electroblotted onto Hybond N+nylon filter (Amersham Biosciences, GE Healthcare Bio-sciences, Piscataway, N.J.). The [γ-³²P]-ATP end-labeled (by Polynucleotide kinase, Roche, Indianapolis, Ind.) oligonucleotide probes for miR-145 were hybridized to the filter in Rapidhyb buffer (Amersham Biosciences, Piscataway, N.J.). The probe, anti-sense oligo against mature miR145 (5′-AAGGGATTCCTGGGAAAACTGGAC) was synthesized by IDT (Integrated DNA Technologies, Coralville, Iowa). Ribosomal RNA (rRNA) 28S, 18S and 5S on the gels stained with ethidium bromide served as loading controls.

Western blots: Cell pellets were collected at different time points (24, 48, and 96 hours post transfection) for protein extraction using RIPA lysis buffer (50 mM Tris-HCl, pH 8.0, 250 mM NaCl, 1% NP40, 0.5% (w/v) sodium deoxycholate, 0.1% SDS and complete mini-protease inhibitors (Roche). BioRad gel (4-15% Tris-HCl, Cat#161-1158) and gel running (Cat#161-0072)/transferring (cat#161-0771) system was used to separate IRS1 proteins, detected by anti-IRS1 polyclonal antibody against IRS-1 (Cell Signaling Technology, Danvers, Mass.). GAPDH served as internal control (Mouse anti-Rabbit GAPDH, Research Diagnostics Inc, Concord Mass.).

Luciferase assay: Dual luciferase vector psiCHECK2 was purchased from Promega (Madison, Wis.). HCT116-Dicer-KO#2 cells were seeded in 96-well plate. The cells were transfected with different psiCHECK2 constructs containing 3′UTR of human IRS1 or miR145 potential binding sites (see supplementary data), in the presence or absence of miR-145 (Dharmacon, Chicago, Ill.). 48 hours later, the firefly and Renilla luciferase activities were assayed using Dual-Glo Luciferase assay system (Promega) in Tecan Safire Microplate Reader U. Because all the miR potential binding sequences were cloned at the 3′ of Renilla Luciferase gene, the ratio of the luminescent signals from Renilla vs. firefly represents the target specificity of miR5. All experiments were performed in triplicate.

Plasmids: The pSuper.retro.neo.GFP plasmid (abbreviated pSuper) was purchased from Oligoengine (www.oligoengine.com). It is controlled by a 5′ LTR, has a variety of restriction sites for insertion, and the transfected cells can be selected either by neomycin or GFP (FACS sorter). It has been tested by Cimmino et al. (35). Double strand-oligo inserts, ˜70 nt hairpin stem-loop pre-miR145 plus 20, 40, 80, or promoter+160 nt flanking sequences at each side of hairpin, were PCR amplified from human genomic DNA (Promega, G3041) and cloned into BglII and HindIII sites of pSuper. The resulting constructs were called pSuper-hairpin145-20 nt (clone #26), pSuper-hairpin145-40 nt (clone #28), pSuper-hairpin145-80 nt (clone #30), and pSuper-hairpin145-160 nt (clone #32). The sequences of the inserts were confirmed by DNA sequencing using primers suggested by OligoEngine (Seattle, Wash.). The mature miR145 (24 nt) was also directly cloned into pSuper. The resulting clones are referred to as pSuper-maturel 45-24 nt (clone #18).

All the above primers for PCR and cloning are listed in Supplemental Material (infra).

The potential binding sites of miR145 on the 3′UTR of human IRS1 were cloned into multiclonal sites (MCS) of a dual luciferase vector psiCHECK2 (Promega, Madison, Wis.). Double strand oligos (listed in Supplemental Material) were generated by annealing sense and antisense strands, and further ligated into psiCHECK2 digested with XhoI and NotI.

The following primers were designed to RT-PCR the 3′UTR of human IRS1 from total RNA extracted from HCT116 cells. This RT-PCR product is about 1 kb, and covers the entire 3′UTR of IRS1 mRNA.

| | |
|---|---|
| XhoI-3UTR primer: | |
| ccgCTCGAGCTCAACTGGACATCACAGCAG | (SEQ ID NO: 3) |
| NotI-3UTR-primer: | |
| ttGCGGCCGCTAAAAGATCAACAGTATCTAGTTTA | (SEQ ID NO: 4) |

The corresponding clones were called psiCHECK2-145site#1 (clone #81), psiCHECK2-145site#2 (clone #83), psiCHECK2-145 sites 1+2 (clone #85), and psiCHECK2-entire3UTR-1 kb (clone #75). The forward and reverse sequencing primers according to psiCHECK2 sequence around MCS were designed and synthesized by IDT to confirm the clones.

The Forward sequencing primer was called hRluc-Fd-1610-1629 (5′-TGCTGAAGAACGAGCAGTAA) (SEQ ID NO: 5) and the reverse primer was called pTK-Rs-1744-1763 (5′-CGAGGTCCGAAGACTCATTT) (SEQ ID NO:6).

Truncated IRS1˜3 kb fragment which contains 5′UTR and a truncated mouse IRS1 gene (about 859 aa residues instead of a full length IRS1 protein, 1232 aa.) was cloned into pcDNA3.1 (Invitrogen, Carlsbad, Calif.). The resulting vector was called pcDNA3.1-truncated mIRS1.

Database. miR target genes were screened with the “Target Scan” program, located at http://genes.mit.edu/targetscan/S2005.html, the miRanda program located at http://www.cbio.mskcc.org/cgi-bin/mirnaviewer/mirnaviewer.pl, the miRBase at http://microrna.sanger.ac.uk/targets/v3 and miRNAMap at http://mirnamap.mbc.nctu.edu.tw. The targets were confirmed by BLAST alignment with the corresponding NCBI DNA database for homologies between miRs and their targets.

### Results

Potential IRS1-specific miR5.

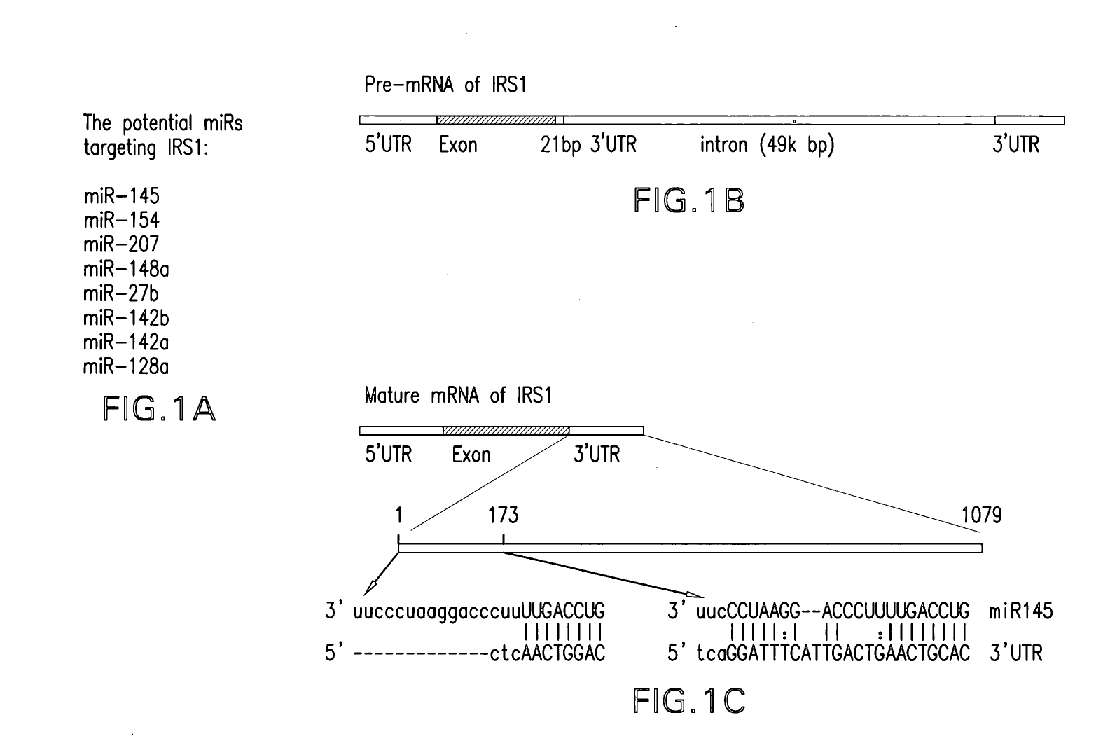

The database identified several miRs as targeting IRS-1, and selected candidates are detailed in FIG. 1, panel A. The structure of the IRS-1 pre-mRNA is unusual and relevant to the experiments described below. The pre-mRNA structure (NCBI for NM-010570, GeneID: 16367. Locus tag: MGI:99454) is presented in panel B. In terms of 3′UTR, the IRS-1 mRNA has an exon of 4,640 bp, with the coding region extending from residue 924 to residue 4619. Then the 3′UTR begins (21 bp), interrupted by an intron of 49,172 bp, and completed by an additional 995 by of 3′UTR. FIG. 1, panel C, gives a more detailed presentation of the 3′UTR of the IRS-1 cDNA, with the two putative binding sites for miR145. One binding site is in the 21 bp sequence immediately after the stop codon, while the 2^{nd} binding site is separated from the first (in the genome) by almost 50,000 bp.

HCT116 and DLD1 cells.

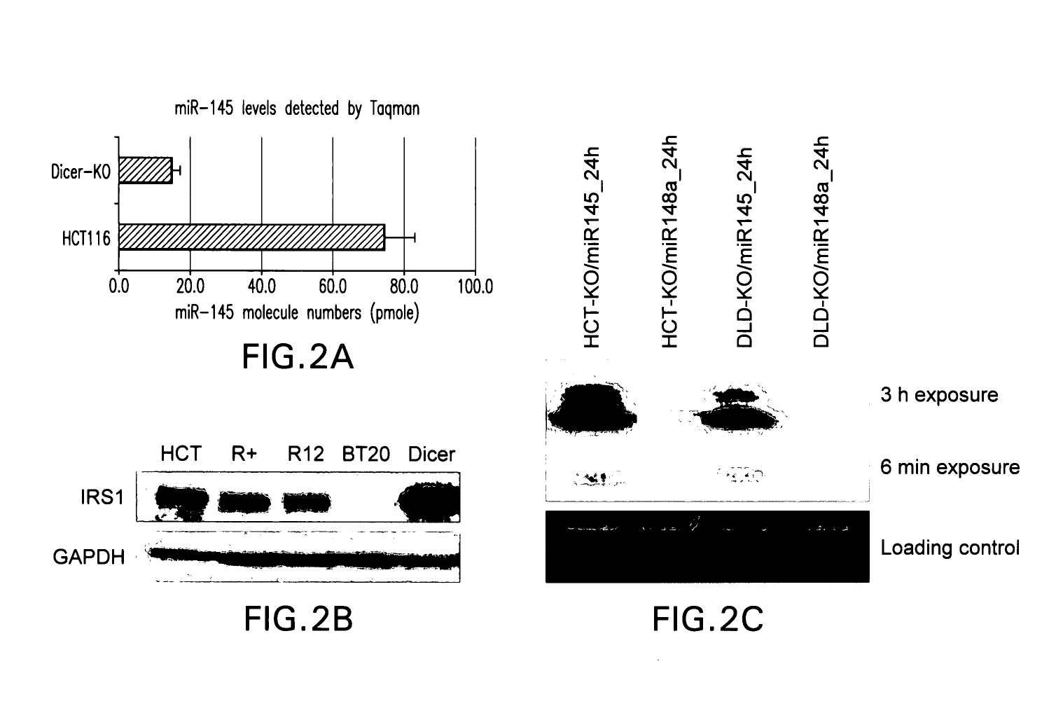

Parental HCT116 and DLD1 cells are derived from colon carcinoma cell lines frequently used in research. HCT116-Dicer-KO and DLD1-KO cells are HCT116 and DLD1 cells in which exon 5 of the Dicer gene (the helicase domain) has been disrupted (33). Because Dicer is required for proper processing of mature miRs, these cells have markedly reduced amounts of mature miRs and display accumulation of miR precursors. The low levels of mature miR145 in HCT116-KO cells, in comparison to parental cells, are shown in FIG. 2, panel A. Actually, miR145 cannot be detected in either parental or HCT116-KO cells by Northern blot (data not shown), and can only be detected by Taqman.

FIG. 2, panel B, shows IRS-1 protein levels in selected cell lines. IRS-1 levels are slightly higher in HCT116-KO cells than in parental HCT116 cells (GAPDH levels monitor protein loading). Included in this western blot were lysates of R+ and R12 cells, mouse embryo fibroblasts known to have substantial levels of IRS-1 (34) as well as BT-20 mammary cancer cells, that do not express IRS-1 at all (8) and serve as the negative control. FIG. 2, panel C, showed that both HCT116 and DLD1 KO cells can be transfected efficiently with a synthetic oligo of miR145 (Dharmacon). miR148a oligos were also transfected into both HCT116-KO and DLD1-KO cells as negative controls for the Northern blot with labeled miR145 probe, (transfected miR148a could be detected after transfection, with the appropriate miR148a probe, data not shown). For other studies, the inventors used mostly HCT116-KO cells, designated as KO cells hereafter, to screen several synthetic oligos for their ability to decrease IRS-1 levels.

miR145 Down-Regulates IRS-1 in KO Cells.

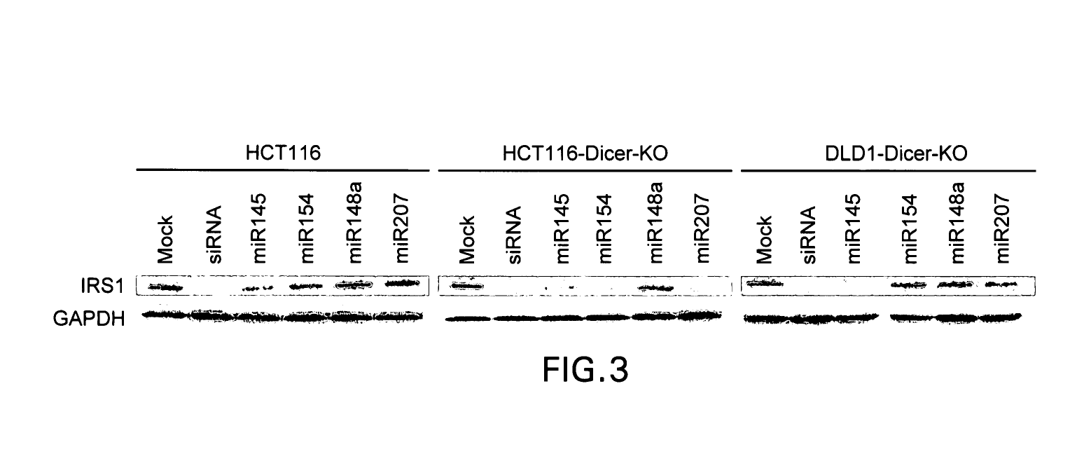

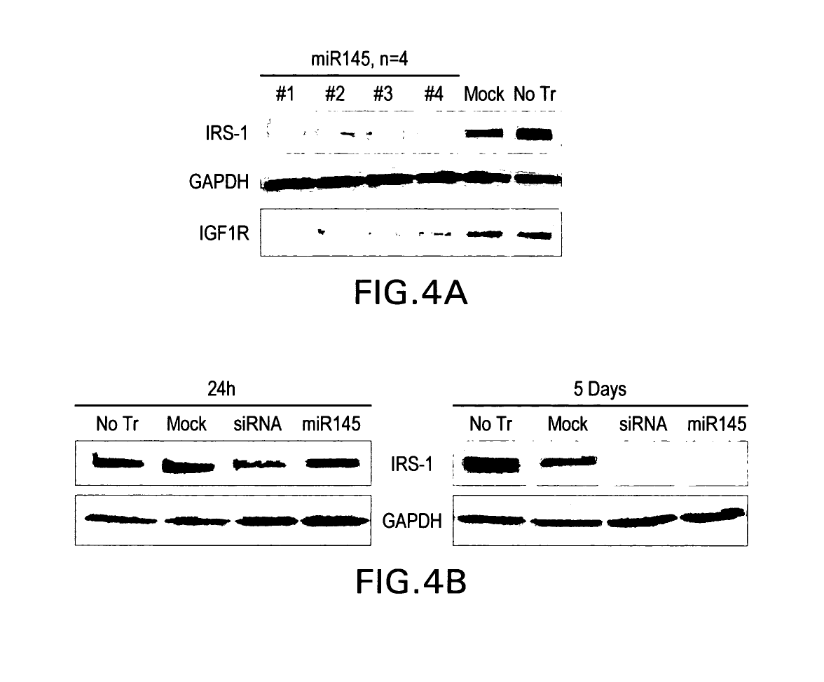

In earlier experiments, the effects of miR oligos at 24 and 48 hrs after transfection were determined. The results appeared to be inconclusive (not shown). As such, the inventors decided to try longer time points, given the half-life of the IRS-1 protein (12). FIG. 3 details that miR145 decreases IRS-1 levels 96 hrs after transfection. miR148a, another miRNA predicted to target IRS-1, failed to decrease IRS-1 levels. Likewise, miR145 and miR207 oligos down-regulated IRS-1 in parental HCT116 cells, KO HCT cells and KO DLD1 cells miR154 was effective only on the first 2 cell lines. An siRNA against IRS-1 (see Methods and Materials) appeared to be effective than miR145 in down-regulating IRS-1 protein levels, especially in parental cells. FIG. 4, panel A, shows repeated experiments in which KO HCT116 cells were transfected with the miR145 oligo, in four separate experiments. In all of them, 96 hrs after transfection, the levels of IRS-1 protein were significantly lower than in untreated or mock-transfected KO cells. miR145 also down-regulated the IGF-IR (FIG. 4, panel A), although to a lesser extent than IRS-1. In this communication, we have focused on miR145 and its targeting of IRS-1.

The effect of miR145 was compared to the effect of siRNA against IRS-1 on IRS-1 levels. This is shown in FIG. 4, panel B (right panel), where IRS-1 levels were measured 24 hrs and 5 days after transfection. While early on, siRNA appeared to be more efficient than miR145 in down-regulating IRS-1 protein levels, its affect on down modulating IRS-1 levels were only marginal at or after 24 hours. (left panel of panel B).

IRS-1 mRNA Levels are not Down-Regulated by miR145.

It is generally agreed that in the majority of cases, miRs act by inhibiting translation, although in some cases, they may cause breakdown of the mRNA (see Introduction). To test this hypothesis, the levels of IRS-1 mRNA in KO cells transfected with either miR145 oligos or siRNA/IRS 1 were tested, and compared to control cells (untreated or mock-transfected). The results (by TaqMan real-time-RT-PCR) of repeated experiments are summarized in FIG. 5. As expected, siRNA markedly decreased IRS-1 levels, but these remain constant in cells transfected with miR145 synthetic oligos. In fact, there was a small but reproducible increase of IRS-1 mRNA in cells transfected with the miR oligo. These experiments demonstrate that miR145 down-regulates the IRS-1 protein, but not the mRNA.

miR145 Down-Regulates a Reporter Gene with Sequences from the 3′UTR of IRS-1.

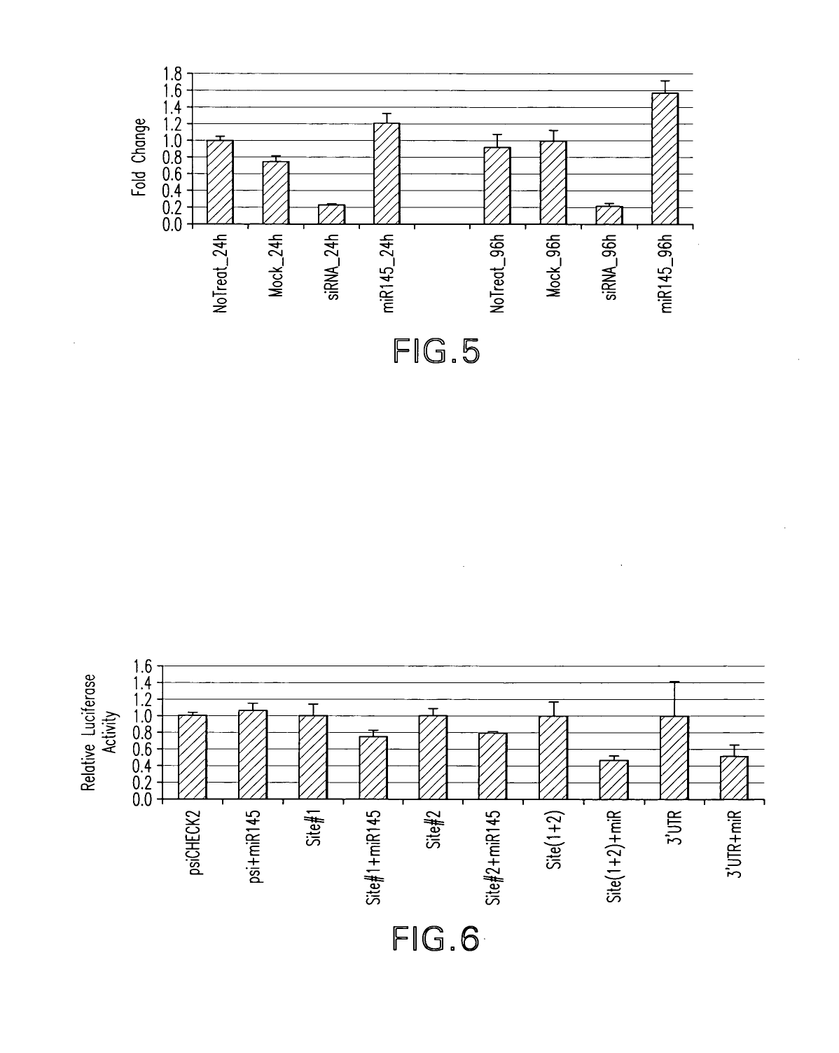

To confirm that miR 145 targets the 3′UTR of IRS-1, experiments were carried out to determine the specificity of the 3′UTR targeting (36-38). The general approach has been to insert the 3′UTR in question at the 3′ end of a reporter gene, often luciferase (36). Following the general approach and protocol, four different constructs in which luciferase was expressed with the 3′UTR of IRS-1 (full length) or with the presumed binding sites of miR145 to the 3′UTR of IRS-1 cDNA (see Methods and Materials) were made. One construct had the 1^{st} putative binding site for miR145, a 2^{nd} construct had the 2^{nd} putative binding site and the final construct had both binding sites (see FIG. 1). The constructs were then co-transfected with miR145 oligos, with cells transfected only with the constructs serving as the controls. The results of a typical experiment are shown in FIG. 6. There was a significant decrease in the expression of the luciferase reporter in cells co-transfected with miR145 oligos and the luciferase carrying the full length 3′UTR of IRS-1 or a 3′UTR containing the two presumed binding sites of 3′UTR for miR145. The constructs in which luciferase had only one binding site for miR145 were also slightly decreased, but to a lesser extent. This experiment was repeated three times, with essentially the same results.

IRS-1 without a 3′UTR.

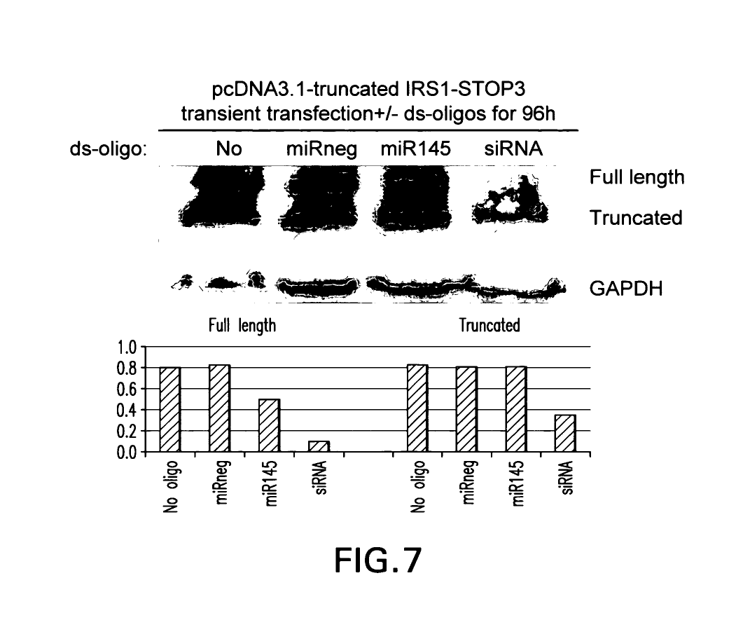

Another way of confirming that a given 3′UTR is targeted by a miR is to ask whether the miR no longer down-regulates a protein, whose cDNA has been deprived of its 3′UTR. To test this hypothesis, the inventors used a truncated IRS-1 cDNA lacking its 3′UTR, and coding for a shorter protein, distinguishable from the wild type endogenous IRS-1 in HCT116 cells. The truncated IRS-1 was transfected with miR145 oligos into Dicer-KO cells and the results (96 hrs after transfection) are summarized in FIG. 7. The endogenous wild type IRS-1 was down-regulated (about 50%) by miR145, but the truncated IRS-1 was not. The siRNA was used as a control to show that both full length and truncated proteins are down-regulated by the siRNA against IRS-1.

Expression of miR145 in pSuper.

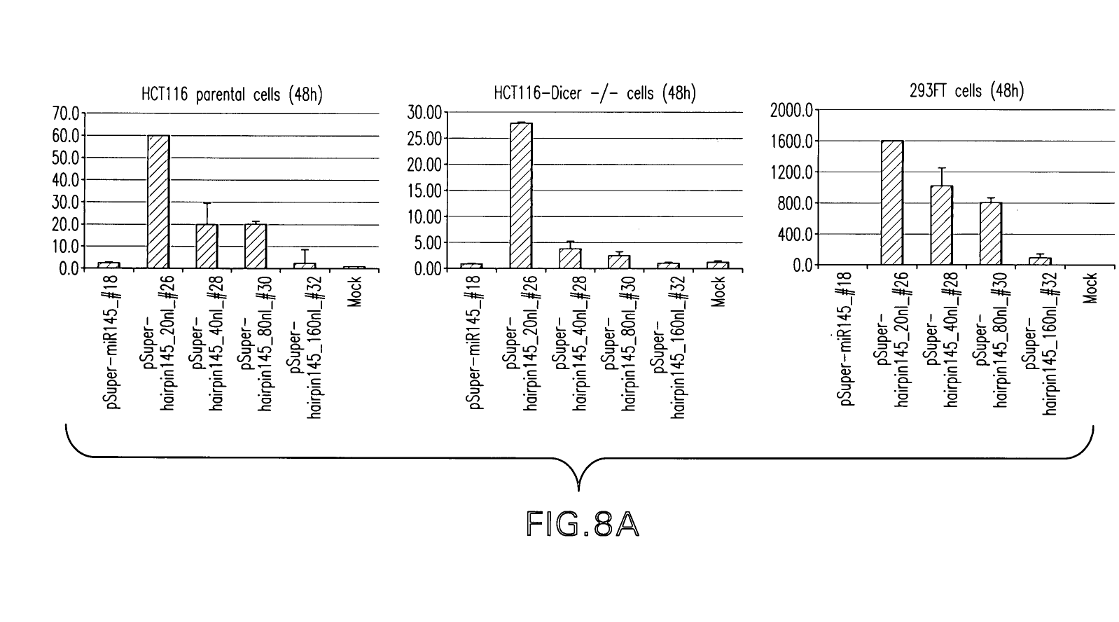

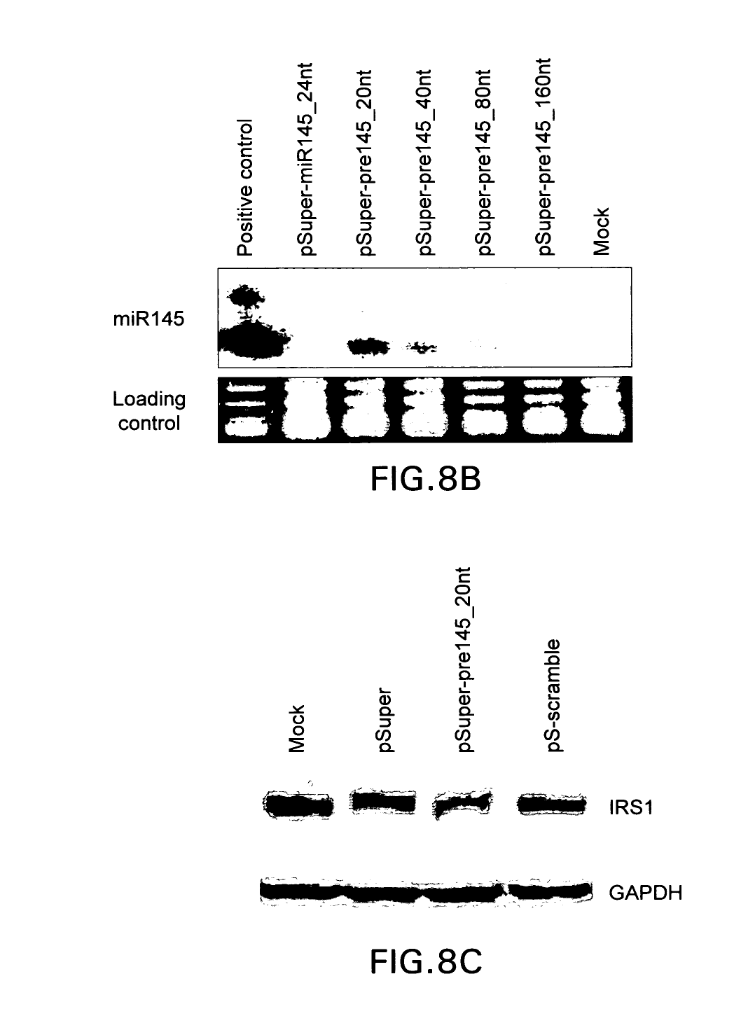

The inventors next attempted to express miR145 in the pSuper plasmid. Preliminary experiments indicated that cloning of the mature miR145 straight into pSuper did not result in detectable expression. The inventors thus investigated whether the addition of flanking sequences to the precursor miR could increase the levels of expression. The flanking sequences used were the genomic sequences flanking the hairpin precursor miR145 (see database). 20, 40 and 80 nucleotides on each side were used. The results (FIG. 8, panel A) show Taqman RT PCR determinations of mature miR145 levels in parental HCT116 cells, KO cells and 293FT cells transfected for 48 hrs with the different pSuper constructs. 20 and 40 nucleotides of flanking sequences improved the expression of miR145 cloned in pSuper, with the 20 nucleotides being the obvious first choice. The experiments were repeated using Northern blots to measure the levels of mature miR145 (FIG. 8, panel B). By these methods, 20 nucleotides of flanking sequences are the optimal condition for miR expression, although some expression is detectable also with 40 and 80 flanking nucleotides. This is more evident in 293FT cells than in parental HCT116 cells. This data varies with the report by Chen et al. (24), who found that the general strategy for miR expression required 270 nucleotides (22 nucleotides of mature miR plus 125 nucleotides of genomic sequences on each side). The discrepancy may be due to the miR or the pSuper. The effect of pSuper/miR145/20 nucleotides on IRS-1 levels in parental HC116 cells wee also tested. IRS-1 was down-regulated in cells transfected with this pSuper construct (Clone #26) 96 hrs after transfection (FIG. 8).

Effect of miR145 and siRNA/IRS1 on the Growth and Morphology of KO Cells.

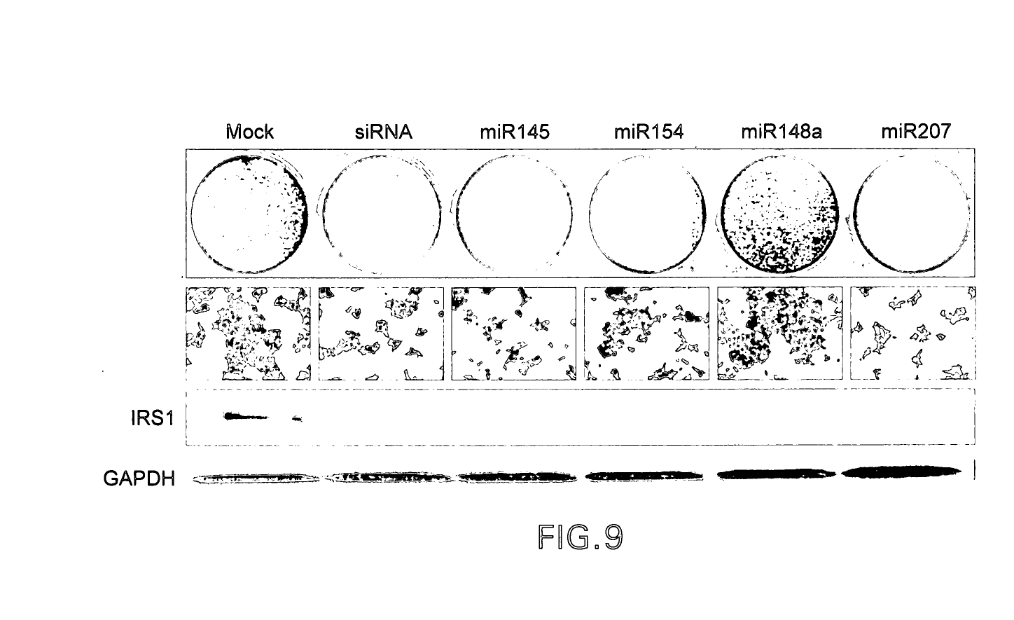

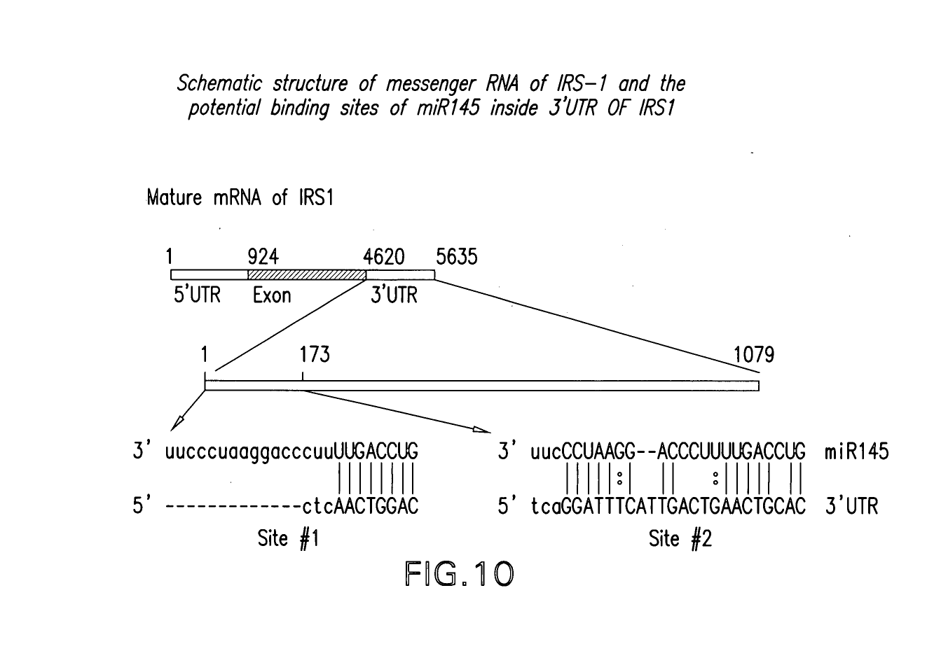

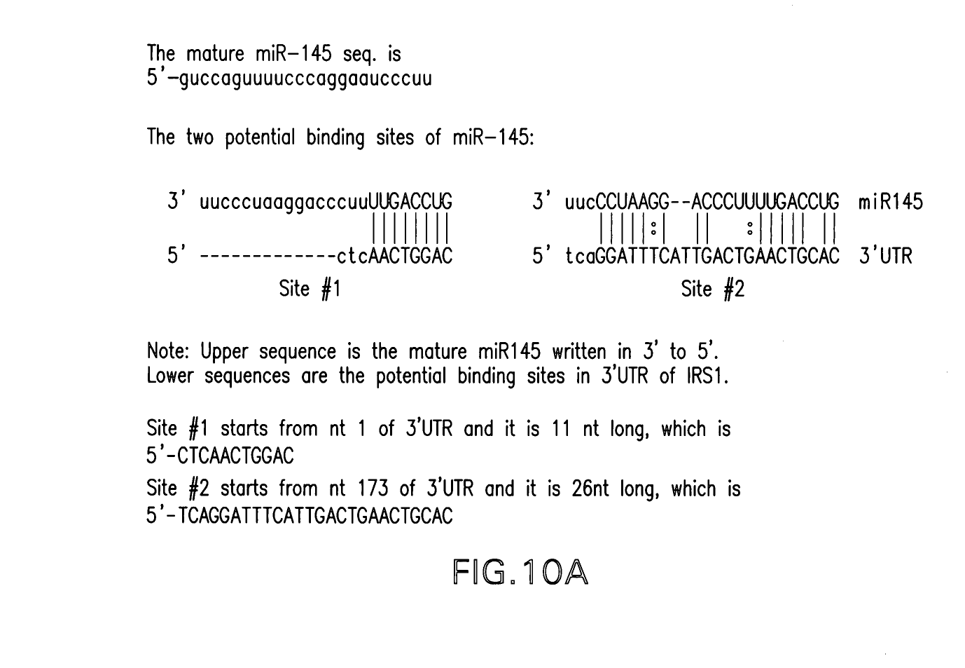

KO cells were transfected with oligos of four different miRs and with siRNA/IRS1 and examined 4 days after transfection. Whether using the plates or the microscopic pictures of the plates (FIG. 9, panels A and B, respectively), it is clear that miR145, miR154 and miR207 inhibit the growth of KO cells as effectively as siRNA. Transfection with miR148a gave the same picture as in mock-tranfected cells. In the experiments described above, while siRNA appeared to present a more effective down-regulation of IRS-1 than the miRs, the consequence, e.g., biological effects, on the other hand, appeared to be very similar (in some experiments, miR145 was even better than the siRNA in inhibiting cell proliferation). It is hypothesized that siRNA may be more effective than miRs in targeting a specific RNA/protein, but miRs are known to have multiple targets, and sometimes these multiple targets involve several proteins on the same signaling pathway (see above).

A careful observation of the treated cells suggests that they may be larger, with somewhat more cytoplasm than the mock-transfected cells. Since IRS-1 is a strong inhibitor of differentiation (7, 39, 40), the inventors inquired whether treatment with the anti-IRS-1 strategies could have induced differentiation of colon cells. After repeated attempts with several markers of differentiation, they were unable to detect differentiation in the miR145-treated cells (data not shown). The biological effects of miR145 were not limited to HCT116 Dicer-KO cells. For example, a dramatic inhibition of cell growth in DLD1 KO cells and in a line of mouse embryo fibroblasts transformed by v-src was also observed (data not shown, but available on request).

### Discussion

While a database search may pique one's interest in investigating a certain hypothesis, such as, for example, the use of miRs to down regulate a specific protein, that by itself is insufficient to demonstrate reduction to practice. Indeed, it is necessary to confirm the target down modulating experimentally. The experimental verification (36-38, 41) is usually based on demonstrating that: 1) the target protein is down-regulated by the predicted miR; 2) a reporter gene expressing the 3′UTR of the targeted mRNA is also down-regulated by the predicted miR; 3) the targeted protein is not down-regulated when the 3′UTR is missing; 4) the miR has a biological function predicted by the biological function of the targeted protein. The present invention aims to satisfy all of the above requirements thereby demonstrating for the first time down-modulation of IRS-1 via the use of miR145 oligoes as a means of inhibiting cancer growth in colon cells. Specifically, the inventors have demonstrated that miR145 oligoes down-regulate the expression of IRS-1 in HCT116 Dicer KO cells, but fail to do so if the 3′UTR of IRS-1 mRNA is missing. A luciferase gene carrying the presumed binding sites of miR145 in the 3′UTR of IRS-1 was shown to be down-regulated by miR145 oligoes. This was true whether luciferase carried the full length 3′UTR of IRS-1, or only the two binding sites predicted by the database. Finally, miR145 oligoes transfected into HCT116-KO cells inhibited their growth, as efficiently as an siRNA against IRS-1.

miR145 was predicted to target IRS-1 mRNA by the database (see FIG. 1). Repeated experiments herein demonstrate that miR145 oligoes down-regulate IRS-1 expression in the KO cells, which express very low levels of mature miR145, undetectable by Northern blots (this study) or micro-arrays (33). The use of the KO HCT116 cells allowed the inventors to screen quickly the more promising miRs and to test the various constructs. The KO cells produced very little amounts of mature miRs (strict Dicer-KO cells are not viable, and for this reason Vogelstein and co-workers generated a cell line with a hypomorphic phenotype. Dicer is ineffective and mature miRs's levels are very low). As to other miRs predicted to down-modulate IRS-1, only miR154 and miR207 demonstrated down-modulation while miR148a did not. This is further proof that mere prediction without more is futile and without utility because not all predictions come true. miR145 also down-regulated the IGF-IR, which was not surprising considering that miRs are said to target multiple mRNAs in the same signaling pathway (31).

The results show that IRS-1 mRNA levels are not affected (while they are strongly affected by an siRNA against IRS-1). Based on the data, miR145 is presumed to act on the translation of IRS-1, which is believed to be the most common mechanism of miR targeting (16). The targeting of the IRS-1 3′UTR was confirmed using a reporter gene, luciferase, carrying the 3′UTR of IRS-1 or its two putative binding sites for miR145.

It is not surprising that the best results on IRS-1 protein down-regulation were obtained 72 or 96 hrs after transfection with miR145 oligos. miR145 does not affect IRS-1 mRNA, and it takes some time for the IRS-1 protein to turn-over (according to Cesarone et al., ref. 12, the half-life of IRS-1 is at least 48 hrs).

The effect of miR145 on the growth and morphology of HCT116 KO cells was dramatic. It is at least as effective in inhibiting growth as the IRS-1 siRNA, although the siRNA is more effective than miR145 in down-regulating IRS-1 protein levels. The explanation may lay again in the observation that miRs usually have multiple targets (see the database) along the same signaling pathway (31, 32). Indeed, in experiments, both the IGF-IR and its docking protein IRS-1 are targeted by miR145. While at first it was hypothesized that inhibition of cell proliferation and morphological changes of HCT116 cells by miR145 treatment might have involved induction of a differentiation pathway, experiments on several differentiation markers for colon cells did not detect any change compared to mock or miR negative control treated cells (not shown, available on request). It seems that the inhibition of HCT116 cells proliferation by miR145 does not involve induction of cell differentiation, and it might be instead the consequence of apoptosis or cell cycle arrest.

miR145 down-regulated IRS1 protein levels but did not decrease the level of IRS1 mRNA. Instead, a slight increase of IRS1 transcripts was observed. Without being bound to a particular theory, it is believed that this is a cause of feedback activity in the cells—when miR145 suppresses the translation of IRS1, cells “see” less IRS1 protein and to compensate, the transcription of irs1 gene is accelerated. The inventors did detect this compensatory increase of IRS1 mRNA level by TaqMan real-time RT-PCR (FIG. 5).

A number of reports have suggested a role of miRs in cancer, see the Introduction and the reviews by Hwang and Mendell (42), or by Esquela-Scherker and Slack (29). There are three reports that miR145 is down-regulated in cancer cells (26, 27, 43), and Kent and Mendell (44), in their review, list miR145 as a tumor suppressor miR. In none of these cases, however, was the targeted mRNA identified. Interestingly, in two of those three references, the down-regulation of miR145 was observed in colon cancer cells (28, 43). Another miR reported to inhibit colon cancer cell growth is let-7 (45). Our results, showing that miR145 inhibits colon cancer cells growth, are compatible with those observations. Indeed, this is the first demonstration of a miR that specifically targets a signal transduction molecule of the IGF-IR/insulin receptor axis and inhibits growth of cancer cells. The effect of miR145 on IRS-1 levels and cancer cell growth is in agreement with the frequent observation that IRS-1 is a strong mitogen and an inhibitor of differentiation (3). IRS-1 over-expression causes transformation (tumor growth in mice) of cells that otherwise would have undergone differentiation (7, 46). IRS-1 is a strong inducer of the ID proteins that inhibit differentiation (39, 40) and DeAngelis et al. (8) have shown that transformation by the SV40 T antigen requires tyrosyl phosphorylation of IRS-1. Dalmay and Edwards (47) suggested that the anti-cancer effect of miR145 may be due to the fact that it targets paxillin. While paxillin is a good target, we propose that IRS-1 may be an even better one. miR145 has 1093 predicted targets in human and 890 in mouse according to miRBase (December, 2006). The 5′ seed region, positions 2-8 of mature miRNA, is conserved in metazoan and plays a key role in target recognition. The large number of target mRNAs down-regulated by miRs has been studied by Lim et al (31) using microarray analysis. A similar concept has been adapted to the off-target effects of siRNA. Although siRNA is designed to be perfectly matched with the on-target mRNA, it can also mediate knockdown of dozens to hundreds of other genes via perfect matches between the hexamer or heptamer seed (positions 2-7 or 2-8 of the antisense strand) of an siRNA and the 3′UTR (but not the 5′UTR or open reading frame) of these off-target genes. Because proteome screens of miR targets and/or siRNA off-targets are not as advanced or extensively available as mRNA microarray analysis, identifying potential targets of translational inhibition is still challenging. A more convincing way to prove a phenotype as the consequence of the knockdown of a specific target by RNAi is to design and apply several siRNAs targeting different regions of the same target mRNA. Even though different siRNAs have different off-target effects, if they all produce the same phenotype by targeting the common “on-target” mRNA, we can draw the conclusion of the correlation of the phenotype with the target gene. In our study, we hypothesized that the malignant growth of colon cell HCT116 is related to the expression of IRS1 in these cells. By identifying and introducing miR145, a down-regulated miR in colorectal cancers, into HCT116 cells, suppression of translation of IRS1 by miR145 leads to the inhibition of cancer cell proliferation. As a proof-of-concept control, siRNA against IRS1 were also transfected into HCT116 cells. Cleavage of IRS1 mRNA by siIRS1 and knockdown of the IRS1 protein again inhibited cell growth. This confirmed the relationship between the phenotype (inhibition of cell proliferation) and the target specificity of miR145 on IRS1. Furthermore, miR negative control or miR-148a treated cells neither changed the levels of IRS1 protein nor suppressed cell proliferation, which again corroborated the hypothesis that phenotypic inhibition of colon cancer cell proliferation is correlated with the down-regulation of the target protein IRS1 via IRS1-specific miR145 or siRNA.

Note however, that this does not exclude the possibility that miR145 also targets other mRNAs of the same signaling pathway.

Although expression of miRs by pSuper has been reported several times in the literature, obtaining good expression was difficult. In fact, contrary to prior reports, the expression of a mature miR145 in pSuper required the presence of 20 genomic flanking nucleotides on each side of the precursor miR145 sequence. This discrepancy with the literature may be specific to miR145.

miR148a was also predicted by the computer database to be a suitable miR for targeting IRS1. However, as demonstrated supra, it failed to down-regulate IRS1 translation nor inhibit colon cancer cell proliferation. Interestingly, a review published recently by Cummins and Velculescu (48) listed differentially expressed miRNAs in colorectal cancer. In this list, while miR145 and miR143 are down-regulated, miR148a was reported to be up-regulated in colorectal adenocarcinomas compared to matched normal colonic epithelia. This coincidence sheds light on the potential usage of miR145 as an anti-colon cancer therapeutic by targeting IRS1.

In conclusion, the inventors have demonstrated that miR145 does indeed target IRS-1 and has a profound biological effect on the growth of human colon cancer cells.

### REFERENCES

1. Baserga, R. (2004) Cell Growth: Control of Cell Size, Cold Spring Harbor Laboratory Press, NY
2. White, M. F. (1998) Mol. Cell. Bioch. 182, 3-11
3. Baserga, R. (2000) Oncogene 19, 5574-5581
4. Chang, Q., Li, Y., White, M. F., Fletcher, J. A., and Xiao, S. (2002) Cancer Res. 62, 6035-6038
5. Sun, X. J., Pons, S., Wang, L. M., Zhang, Y., Yenush, L., Burks, D., Myers, M. G. Jr., Glasheen, E., Copeland, N. G., Jenkins, N. A., and White, M. F. (1997) Mol. Endocr. 11, 251-262
6. Valentinis, B., Romano, G., Peruzzi, F., Morrione, A., Prisco, M., Soddu, S., Cristofanelli, B., Sacchi, A., and Baserga, R. (1999) J. Biol. Chem. 274, 12423-12430
7. Valentinis, B., Navarro, M., Zanocco-Marani, T., Edmonds, P., McCormick, J., Morrione, A., Sacchi, A., Romano, G., Reiss, K., and Baserga, R. (2000) J. Biol. Chem. 275, 25451-25459
8. DeAngelis, T., Chen, J., Wu, A., Prisco, M., and Baserga R. (2006) Oncogene 25, 32-42
9. Dearth, R. K., Cui, X., Kim, H. J., Kuiatse, I., Lawrence, N. A., Zhang, X., Divisova, J., Britton, O. L., Mohsin, S., Allred, D. C., Hadsell, D. L., and Lee, A. V. (2006) Mol. Cell. Biol. 26, 9302-9314
10. D′Ambrosio, C., Keller, S. R., Morrione, A., Lienhard, G. E., Baserga, R., and Surmacz, E. (1995) Cell Growth & Diff. 6, 557-562
11. Del Rincon, S. V., Guo, Q., Morelli, C., Shiu, H. Y., Surmacz, E., and Miller, W. H. (2004) Oncogene 23, 9269-9279
12. Cesarone, G., Garofalo, C., Abrams, M. T., Igoucheva, O., Alexeev, V., Yoon, K., Surmacz, E., and Wickstrom, E. (2006) J. Cell. Biochem. 98, 440-450
13. Bohni, R., Riesco-Escovar, J., Oldham, S., Brogiolo, W., Stocker, H., Andruss, B. F., Beckingham, K., and Hafen, E. (1999) Cell 97, 865-875
14. Pete, G., Fuller, G. R., Oldham, J. M., Smith, D. R., D′Ercole, A. J., Kahn, C. R., and Lund, P. K. (1999) Endocrinology 140, 5478-5487
15. Sun, H., Tu, X., and Baserga, R. (2006) Cancer Res., in press
16. Lee, R. C., and Ambros, V. (2001) Science 294, 862-864
17. Ke, X. S., Liu, C. M., Liu, D. P., and Liang, C. C. (2003) Current Opin. Chem. Biol. 7, 516-523
18. Lai, E. C. (2003) Current Biol. 13, R925-R936
19. Zeng, Y. (2006) Oncogene 25, 6156-6162
20. Lee, Y., Kim, M., Han, J., Yeom, K. H., Lee, S., Baek, S. H., and Kim, V. N. (2004) EMBO J. 23: 4051-4060
21. Lee, Y., Ahn, C., Han, J., Choi, H., Kim, J., Yim, J., Lee, J., Provost, P., Radmark, O., Kim, S., and Kim, V. N. (2003) Nature 425, 415-419
22. Krichevsky, A. M., King, K. S., Donahue, C. P., Khrapko, K., and Kosik, K. S. (2003) RNA 9, 1274-1281
23. Fahr, K. K., Grimson, A., Jan, C., Lewis, B. P., Johnston, W. K., Lim, L. P., Burge, C. B., and Bartel, D. P. (2005) Science 310, 1817:1821
24. Chen, C. Z., Li, L., Lodish, H. F., and Bartel, D. P. (2004) Science 303, 83-86
25. Shi, B., Prisco, M., Calin, G., Liu, C-g., Russo, G., Giordano, A., and Baserga R. (2006) J. Cell. Physiol. 207, 706-710
26. Michael, M. Z., O′Connor, S. M., van Host Pellekaan, N. G., Young, G. P., and James, R. J. (2003) Mol. Cancer. Res. 1, 882-891
27. Iorio, M. V., Ferracin, M., Liu, C. G., Verones, A., Spizzo, R., Sabbioni, S., Magri, E., Pedriali, M., Fabbri, M., Campiglio, M., Menard, S., Palazzo, J. P., Rosenberg, A., Musiani, P., Volinia, S., Nenci, I., Calin, G. A., Negrini, M., and Croce, C. M. (2005) Cancer Res. 65, 7065-7070
28. Calin, G. A., and Croce, C. M. (2006) Cancer Res. 66, 7390-7394
29. Esquela-Kerscher, A., and Slack, F. J. (2006) Nature Rev. Cancer 6:259-269
30. Akao, Y., Nakagawa, Y., and Naoe, T. (2006) Oncol. Rep. 16, 845-850
31. Lim, L. P., Lau, N.C., Garrett-Engele, P., Grimson, A., Schelter, J. M., Castle, J., Bartel, D. P., Linsley, P. S., and Johnson, J. M. (2005) Nature 433, 769-773
32. Cui, Q., Yu, Z., Purisima, E. O., and Wang, E. (2006) Mol. Systems. Biol. doi:10.138/msb4100089
33. Cummins, J. M., He, Y., Leary, R. J., Pagliarini, R., Diaz, L. A., Jr., Sjoblom, T., Barad, O., Bentwich, Z., Szafranska, A. E., Labourier, E., Raymond, C. K., Roberts, B. S., Juhl, H., Kinzler, K. W., Vogelstein, B., and Velculescu, V. E. (2006) Proc. Natl. Acad. Sci. 103, 3687-3692
34. Reiss, K., Valentinis, B., Tu, X., Xu, S. Q., and Baserga, R. (1998) Exp. Cell Res. 242, 361-372
35. Cimmino, A., Calin, G. A., Fabbri, M., Iorio, M. V., Ferracin, M., Shimizu, M., Wojcik, S., Aqeilan, R. I., Zupo, S., Dono, M., Rassenti, L., Alder, H., Volinia, S., Liu, C., Kipps, T. J., Negrini, M., and Croce, C. M. (2005) Proc. Natl. Acad. Sci. 102, 13944-13949
36. Wu, L., and Belasco, J. G. (2005) Mol. Cell. Biol. 25, 9198-9208
37. Fazi, F., Rosa, A., Fatica, A., Gelmetti, V., DeMarchis, M. L., Nervi, C., and Bozzoni, I. (2005) Cell 123, 819-831
38. Martin, M. M., Lee, E. J., Buckenberger, J. A., Schmittgen, T. D., and Elton, T. S. (2006) J. Biol. Chem. 281, 18277-18284
39. Belletti, B., Prisco, M., Morrione, A., Valentinis, B., Navarro, M., and Baserga, R. (2001) J. Biol. Chem. 276, 13867-13874
40. Prisco, M., Peruzzi, F., Belletti, B., and Baserga, R. (2001) Mol. Cell. Biol. 21, 5447-5458
41. Krutzfeldt, J., Poy, M. N., and Stoggel, M. (2006) Nature Genet. 38, 514-5,4-519
42. Hwang, H-W, and Mendell, J. T. (2006) Brit. J Cancer 94, 776-780
43. Bandres, E., Cubedo, E., Agirre, X., Malumbres, R., Zarate, R., Ramirez, N., Abajo, A., Navarro, A., Moreno, I., Monzo, M., and Garcia-Foncillas, J. (2006) Mol. Cancer. 5, 29-39
44. Kent, O. A., and Mendell, J. T. (2006) Oncogene 25, 6188-6196
45. Akao, Y., Nakagawa, Y., and Naoe, T. (2006) Biol. Pharm. Bull. 29, 903-906
46. Cristofanelli, B., Valentinis, B., Soddu, S., Rizzo, M. G., Marchetti, A., Bossi, G., Morena, A. R., Dews, M., Baserga, R., and Sacchi, A. (2000) Oncogene 19, 3245-3255
47. Dalmay, T., and Edwards, D. R. (2006) Oncogene 25, 6170-6175

### Supplemental Data:

1. Primers for PCR to amplify hairpin stem-loop precursor mir-145 plus different flanking sequence from human genomic DNA.
Strategy#1: 20 nt at both sides:

| | |
|---|---|
| #1_BglII-Fd primer: | |
| 5′-GGAAGATCTCGCTGAAGGCCACTCGCTCC | (SEQ ID NO: 3) |
| #1_HindIII-Rs primer: | |
| 5′-CCCAAGCTTGGAGGCAAATCCAGCTGTGA | (SEQ ID NO: 4) |

The resulting clones are called pSuper-hairpin145-20 nt (clone #26).

Strategy#2: 40 nt at both sides:

| | |
|---|---|
| #2_BglII-Fd primer: | |
| 5′-GGAAGATCTTAGGGACACGGCGGCCTTGG | (SEQ ID NO: 5) |
| #2_HindIII-Rs primer: | |
| 5′-CCCAAGCTTGGGCAACTGTGGGGTGGGAA | (SEQ ID NO: 6) |

The resulting clones are called pSuper-hairpin145-40 nt (clone #28).

Strategy#3: 80 nt at both sides:

| | |
|---|---|
| #3_BglII-Fd primer: | |
| 5′-GGAAGATCTAGAGAACTCCAGCTGGTCCT | (SEQ ID NO: 7) |
| #3_HindIII-Rs primer: | |
| 5′-CCCAAGCTTCCAGCCGAGGCCCCATTGGG | (SEQ ID NO: 8) |

The resulting clones are called pSuper-hairpin145-80 nt (clone #30).

Strategy#4: predicted endogenous promoter of human miR145 included:

Primers were designed to PCR hairpin pre-mir-145 from human genomic DNA including the predicted promoter at 3′ and 160 bp at 5′ of pre-miR145 sequence.

| | |
|---|---|
| #4_BglII-Fd primer: | |
| 5′-GGAAGATCTATCTGCCTTCAAATCCATGT | (SEQ ID NO: 9) |
| #4_HindIII-Rs primer: | |
| 5′-CCCAAGCTTATAGACACGATGGAAAGAAA | (SEQ ID NO: 10) |

The resulting clones are called pSuper-hairpin145_{—}160 nt (clone #32).

Strategy#5: The mature miR145 (24 nt) was also directly cloned into pSuper by annealing the ton and bottom strands of the following sequences synthesized by IDT.

| | |
|---|---|
| pSuper_Top oligo: | |
| 5′-GATCTGTCCAGTTTT CCCAGGAATCCCTTA | (SEQ ID NO: 11) |
| pSuper_Bottom: | |
| 5′-AGCTTAAGGGATTCCTGGGAAAACTGGACA | (SEQ ID NO: 12) |

The resulting clones are called pSuper-mature145-24 nt (clone #18).

The potential binding sites of miR145 on 3′UTR of human IRS1 were cloned into multi-clonal sites (MCS) of a dual luciferase vector psiCHECK2 (Promega). The top and bottom strands for each binding site (miR145 site #1 or #2) or both (miR145 sites(1+2)) with XhoI and NotI at 5′ and 3′ end respectively are listed as follows:

miR145 site #1_Sense:

(SEQ ID NO: 13)

TCGAGAGCCAGAGGACCGTCAGTAGCTCAACTGGACATCACAGCAGAATG
AAGACCGC
miR145 site#1_AntiSense:

(SEQ ID NO: 14)

GGCCGCGGTCTTCATTCTGCTGTGATGTCCAGTTGAGCTACTGACGGTCC
TCTGGCTC
miR145 site#2_Sense:

(SEQ ID NO: 15)

TCGAGTTTACTTTATCCAATCCTCAGGATTTCATTGACTGAACTGCACGT
TCTATATTGTGCCAGC
miR145 site #2_AS:

(SEQ ID NO: 16)

GGCCGCTGGCACAATATAGAACGTGCAGTTCAGTCAATGAAATCCTGAGG
ATTGGATAAAGTAAAC
miR145 site (1 + 2)_Sense:

(SEQ ID NO: 17)

TCGAGCCAATCCTCAGGATTTCATTGACTGAACTGCACGTTCTATATGTG
CCAACTCAACTGGACATCACC
miR145 site (1 + 2)_AS:

(SEQ ID NO: 18)

GGCCGCGTGATGTCCAGTTGAGTTGGCACATATAGAACGTGCAGTTCAGT
CAATGAAATCCTGAGGATTGC

The synthesized sense and anti-sense oligos were annealed to form double-strand oligos and cloned into psiCHECK2 cut with XhoI and NotI.

The following primers were designed to RT-PCR the 3′UTR of human IRS1 from total RNA extracted from HCT116 cells. This RT-PCR product is about 1 kb, which covers the entire 3′UTR of IRS1 mRNA.

XhoI-3UTR primer:

(SEQ ID NO: 19)

CCGCTCGAGCTCAACTGGACATCACAGCAG
NotI-3UTR-primer:

(SEQ ID NO: 20)

TTGCGGCCGCTAAAAGATCAACAGTATCTAGTTTA

The corresponding clones were called psiCHECK2-145site#1 (clone #81), psiCHECK2-145site#2 (clone #83), psiCHECK2-145site(1+2) (clone #85), and psiCHECK2-entire3UTR-1 kb (clone #75).

| | |
|---|---|
| miR-145: | |
| 5′-GUCCAGUUUUCCCAGGAAUCCCUU | (SEQ ID NO: 1) |

The precursor miR-145 is 88 nt hairpin structure. The sequence for hsa-mir-145 precursor is as follows:

(SEQ ID NO: 2)

5′CACCUUGUCCUCACGGUCCAGUUUUCCCAGGAAUCCCUUAGAUGCUAA
GAUGGGGAUUCCUGGAAAUACUGUUCUUGAGGUCAUGGUU

See the web link also:

http://microrna.saner.ac.uk/cgi-bin/sequences/mirna_entry.pl?acc=MI0000461

1. (canceled)

2. A method of diagnosing whether a subject has, or is at risk for developing, a cancer associated with low expression of miR-145 relative to normal in a subject, comprising: (1) reverse transcribing RNA from a test sample obtained from the subject to provide a target oligodeoxynucleotide; (2) hybridizing the target oligodeoxynucleotide to a miRNA-specific probe oligonucleotideto provide a hybridization profile for said test sample; and (3) comparing the test sample hybridization profile to a hybridization profile generated from a control sample, wherein an alteration in the signal is indicative of the subject either having, or being at risk for developing, the cancer.

3. A method of treating subject suffering from colon cancer comprising administering to said subject a nucleic acid molecule sufficient to down regulate expression of an endogenous gene associated with said colon cancer.

4. The method according to claim 3, wherein said endogenous gene is IRS-1.

5. (canceled)

6. The method according to claim 3 wherein said nucleic acid molecule comprises a sequence of nucleotides as set forth in one of SEQ ID NOS: 1 or 2.


SEQUENCE LISTING (20 sequences)

SEQ ID NO: 1 - RNA, 24 nt, Artificial Sequence
  Synthetic nucleotide sequence
guccaguuuucccaggaaucccuu

SEQ ID NO: 2 - RNA, 88 nt, Artificial Sequence
  Synthetic nucleotide sequence
caccuuguccucacgguccaguuuucccaggaaucccuuagaugcuaagauggggauuccuggaaauacuguucuugaggucaugguu

SEQ ID NO: 3 - DNA, 29 bp, Artificial Sequence
  Synthetic nucleotide sequence
ggaagatctcgctgaaggccactcgctcc

SEQ ID NO: 4 - DNA, 29 bp, Artificial Sequence
  Synthetic nucleotide sequence
cccaagcttggaggcaaatccagctgtga

SEQ ID NO: 5 - DNA, 29 bp, Artificial Sequence
  Synthetic nucleotide sequence
ggaagatcttagggacacggcggccttgg

SEQ ID NO: 6 - DNA, 29 bp, Artificial Sequence
  Synthetic nucleotide sequence
cccaagcttgggcaactgtggggtgggaa

SEQ ID NO: 7 - DNA, 29 bp, Artificial Sequence
  Synthetic nucleotide sequence
ggaagatctagagaactccagctggtcct

SEQ ID NO: 8 - DNA, 29 bp, Artificial Sequence
  Synthetic nucleotide sequence
cccaagcttccagccgaggccccattggg

SEQ ID NO: 9 - DNA, 29 bp, Artificial Sequence
  Synthetic nucleotide sequence
ggaagatctatctgccttcaaatccatgt

SEQ ID NO: 10 - DNA, 29 bp, Artificial Sequence
  Synthetic nucleotide sequence
cccaagcttatagacacgatggaaagaaa

SEQ ID NO: 11 - DNA, 30 bp, Artificial Sequence
  Synthetic nucleotide sequence
gatctgtccagttttcccaggaatccctta

SEQ ID NO: 12 - DNA, 30 bp, Artificial Sequence
  Synthetic nucleotide sequence
agcttaagggattcctgggaaaactggaca

SEQ ID NO: 13 - DNA, 58 bp, Artificial Sequence
  Synthetic nucleotide sequence
tcgagagccagaggaccgtcagtagctcaactggacatcacagcagaatgaagaccgc

SEQ ID NO: 14 - DNA, 58 bp, Artificial Sequence
  Synthetic nucleotide sequence
ggccgcggtcttcattctgctgtgatgtccagttgagctactgacggtcctctggctc

SEQ ID NO: 15 - DNA, 66 bp, Artificial Sequence
  Synthetic nucleotide sequence
tcgagtttactttatccaatcctcaggatttcattgactgaactgcacgttctatattgtgccagc

SEQ ID NO: 16 - DNA, 66 bp, Artificial Sequence
  Synthetic nucleotide sequence
ggccgctggcacaatatagaacgtgcagttcagtcaatgaaatcctgaggattggataaagtaaac

SEQ ID NO: 17 - DNA, 71 bp, Artificial Sequence
  Synthetic nucleotide sequence
tcgagccaatcctcaggatttcattgactgaactgcacgttctatatgtgccaactcaactggacatcacc

SEQ ID NO: 18 - DNA, 71 bp, Artificial Sequence
  Synthetic nucleotide sequence
ggccgcgtgatgtccagttgagttggcacatatagaacgtgcagttcagtcaatgaaatcctgaggattgc

SEQ ID NO: 19 - DNA, 30 bp, Artificial Sequence
  Synthetic nucleotide sequence
ccgctcgagctcaactggacatcacagcag

SEQ ID NO: 20 - DNA, 35 bp, Artificial Sequence
  Synthetic nucleotide sequence
ttgcggccgctaaaagatcaacagtatctagttta